(19) United States
(12) Patent Application Publication
(10) Pub. No.: US 2018/0221263 A1
(43) Pub. Date: Aug. 9, 2018

(54) MEDICAMENT

(71) Applicant: Symrise AG, Holzminden (DE)

(72) Inventors: Gabriele Vielhaber, Paris (FR); Marielle Le Maire, Boulogne Billancourt (FR)

(21) Appl. No.: 15/888,433

(22) Filed: Feb. 5, 2018

Related U.S. Application Data
(62) Division of application No. 14/892,109, filed on Nov. 18, 2015, now abandoned.

(30) Foreign Application Priority Data
Feb. 21, 2013 (EP) 13156181.3

(51) Int. Cl. A61K 8/49; A61K 8/40; A61K 8/44; A61K 8/60; A61K 8/67; A61K 8/73; A61K 8/81; A61K 8/86; A61K 8/891; A61Q 19/08; A61K 8/92; A61Q 5/00; A61Q 19/00; A61K 8/26; A61K 8/29; A61K 8/31; A61K 8/34; A61K 8/35; A61K 8/36; A61K 8/37; A61Q 17/04; A61K 8/41

(52) U.S. Cl. CPC A61K 8/4946; A61K 8/40; A61K 8/415; A61K 8/44; A61K 8/4966; A61K 8/60; A61K 8/678; A61K 8/73; A61K 8/8158; A61K 8/86; A61K 8/891; A61Q 19/08; A61K 2800/592; A61K 8/922; A61Q 5/00; A61Q 19/004; A61K 8/26; A61K 8/29; A61K 8/31; A61K 8/345; A61K 8/35; A61K 8/36; A61K 8/375; A61K 2800/782; A61Q 17/04

(57) ABSTRACT

Proposed is a drug containing
(a) at least one compound of Formula (I)

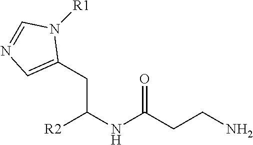

provided that R1 denotes H or CH₃ and R2 denotes H or COOH, or salts thereof, and
(b) at least one UV protection factor
   for the inhibition of MMP-1 expression induced by UV radiation.

### FIELD OF THE INVENTION

The invention lies on the boundary between the fields of pharmaceuticals and cosmetics, and concerns on the one hand a drug for the inhibition of MMP-1 expression induced by UV radiation and on the other a synergistic active ingredient mixture for use as cosmetic agent, particularly as a sun protection agent.

### PRIOR ART

Efforts have been made for some time to prevent or at least delay skin aging. Science has been able to identify a wide variety of accelerators such as harmful substances from the environment, free radicals, oxidative stress, inflammations, ultraviolet radiation (UV radiation), and infrared radiation (IR radiation) as causes of skin aging.

Various mechanisms of skin aging are also being discussed, with enzymatic mechanisms frequently being the topic of discussion and the enzyme “matrix metalloproteinase 1” (MMP-1) being considered particularly significant. Increased expression or activation of MMP-1 or an increase in MMP-1 activity are generally considered to accelerate skin aging.

UV radiation is considered a significant accelerator of skin aging, and its effect is derived from a wide variety of mechanisms, for example from damage to the genetic material, protein denaturation, or induction of MMP-1 expression. Although there have been quite favorable possibilities for reducing skin aging caused by UV radiation up to now, such as the use of UV protection factors, there is an ongoing need to improve the efficacy of such substances, either by developing new filters or providing synergistic combinations of active ingredients.

In this connection, we refer to WO 2010124817 A2 (KAO). The document concerns preparations containing dipeptides. In Examples 14 to 16, hair care agents are disclosed that also contain L-carnosine, but no light protection factors.

Multiple emulsions containing silicone compounds that can optionally also contain carnitine or carnosine are the subject matter of DE 10300782 A1 (BEIERSDORF). These preparations do not contain any additional light protection factors.

WO 2007122822 A1 (SHISEIDO) concerns cosmetic sun protection emulsions. Examples 22 and 23 disclose formulations that also contain carnosine together with other light protection factors such as 5 wt % octocrylene. However, this document does not mention the property of carnosine of inhibiting the expression of MMP-1.

Cosmetic and pharmaceutical preparations containing an antioxidant that protects the skin from IR radiation are known from EP 1591105 A1 (STADA). Suitable antioxidants also include carnosine and anserine.

Preparations that also protect the skin from damage by IR radiation and contain plant extracts, vitamins, ruby powder, mica, and titanium dioxide are the subject matter of EP 2545898 A1 (COTY). In paragraph [0026], carnosine is mentioned as a further suitable auxiliary.

EP 1310238 A2 (BASF) concerns cosmetic or dermatological light protection preparations that contain special monomers or polymers in combination with UV protection factors. Further antioxidants such as carnosine can also be present.

The primary object of the present invention was therefore to reduce or prevent skin aging caused by UV radiation, to describe corresponding synergistic mixtures of active ingredients for this purpose, and to provide special formulations for targeted application of the active ingredients.

A further partial object was to develop preparations that are not only highly efficient but also show highly favorable dermatologic tolerability, and particularly do not cause reddening, bleaching, or tanning of the skin, are non-irritating, do not dry out or chap the skin, and do not leave a moist, scaly, powdery, or sticky film on the skin.

In addition to targeted transport to the site of action, the novel preparations according to the invention are also intended to ensure that the active ingredients according to the invention rapidly reach the site of action after application and continue to be released at the site of action for long periods.

### DESCRIPTION OF THE INVENTION

The subject matter of the invention is a drug containing
(a) at least one compound of Formula (I)

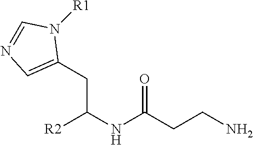

provided that R1 denotes H or CH₃ and R2 denotes H or COOH or salts thereof, and
(b) at least one UV protection factor
   for the inhibition of MMP-1 expression induced by UV radiation.

Surprisingly, it was found that mixtures of the compounds of Formula (I), particularly carnosine, in combination with UV protection factors, provide synergistically improved inhibition of MMP-1 expression and thus improve the performance of conventional UV protection factors in an unexpected manner.

In addition to being highly effective, the active ingredients are well tolerated, not causing reddening, bleaching, or tanning of the skin, are non-irritating, do not dry out the skin, do not form a moist, scaly, powdery, or sticky film, and do not chap the skin. Moreover, these substances are characterized in that when applied topically, they remain at the site of action for long periods, thus ensuring improved UV protection over a satisfactorily long period of time.

Active Ingredients

The active ingredients of Formula (I) are basically known compounds that can be obtained by means of the ordinary processes of organic chemistry. Preferably, this is a group of substances composed of carnosine, L-carnosine, D-carnosine, D/L-carnosine, carnitine, carnitine*HCl, anserine, D-anserine, L-anserine, as well as L-anserine*HNO₃ and mixtures thereof.

According to the invention, salts of the compounds of Formula (I) are understood to preferably be salts of the compounds of Formula (I) with mineral acids, and particularly preferably salts of Formula (II):

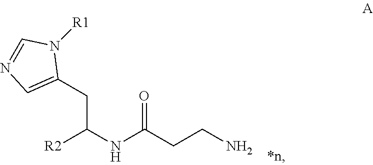

where n denotes 1, 2, or 3, A denotes HCl or HNO₃, R1 denotes H or CH3, and R2 denotes H or COOH.

In preferred embodiments of the invention, the compounds of Formula (I) or salts thereof are used for inhibition of the following processes induced by UV radiation, particularly UV-A radiation:
synthesis of the mRNA coding for MMP-1 or
translation of MMP-1 from the mRNA or
MMP-1 activation,
   wherein the inhibition preferably takes place in or on the fibroblasts of the dermis (dermal fibroblasts).

UV Protection Factors

Mixtures of compounds of Formula (I) or salts thereof with UV protection factors provide synergistic enhancement of protection of the skin and hair against the harmful effects of sunlight. These UV protection factors can be UV-A filters, UV-B filters, pigments, or mixtures thereof that are further explained below.

UV-A and UV-B Protection Filters

UV protection factors are understood to refer, for example, to organic substances that are liquid or crystalline at room temperature (light protection filters) and are capable of absorbing ultraviolet radiation and releasing the absorbed energy in the form of long-wave radiation such as heat. Ordinarily, UV protection factors are contained in amounts of 0.1 to 5 and preferably 0.2 to 1 wt %. UVB filters can be oil-soluble or water-soluble. Examples of suitable oil-soluble substances include:
3-benzylidene camphor or 3-benzylidene norcamphor and derivatives thereof, such as 3-(4-methylbenzylidene)camphor;
4-aminobenzoic acid derivatives, preferably 4-(dimethylamino)benzoic acid-2-ethyl-hexyl ester, 4-(dimethylamino)benzoic acid-2-octyl ester, and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, and 2-cyano-3,3-phenylcinnamic acid-2-ethylhexyl ester (octocrylene);
esters of salicylic acid, preferably salicylic acid-2-ethylhexyl ester, salicylic acid-4-isopropyl benzyl ester, and salicylic acid homomenthyl ester;
benzophenone derivatives, preferably 2-hydroxy-4-methoxybenzophenone, 2-hydroxy-4-methoxy-4′-methylbenzophenone, and 2,2′-dihydroxy-4-methoxybenzophenone;
esters of benzylmalonic acid, preferably 4-methoxybenzylmalonic acid di-2-ethylhexyl ester;
triazine derivatives such as 2,4,6-trianilino-(p-carbo-2′-ethyl-1′-hexyloxy)-1,3,5-triazine and octyl triazone or dioctyl butamidotriazone (Uvasorb® HEB);
propane-1,3-diones such as 1-(4-tert-butyl phenyl)-3-(4′-methoxyphenyl)propane-1,3-dione; and
ketotricyclo (5.2.1.0) decane derivatives.

Examples of suitable water-soluble substances include:
2-phenylbenzimidazole-5-sulfonic acid and alkali, alkaline earth, ammonium, alkylammonium, alkanolammonium, and glucammonium salts thereof;
1H-benzimidazole-4,6-disulfonic acid, 2,2′-(1,4-phenylene)bis-disodium salt (Neo Heliopan® AP);
sulfonic acid derivatives of benzophenones, preferably 2-hydroxy-4-methoxybenzophenone-5-sulfonic acid and salts thereof;
sulfonic acid derivatives of 3-benzylidene camphor such as 4-(2-oxo-3-bornylidene methylbenzene sulfonic acid, 2-methyl-5-(2-oxo-3-bornylidene)sulfonic acid, and salts thereof.

Typical examples of particularly suitable UV-A filters include benzoyl methane derivatives such as 1-(4′-tert-butylphenyl)-3-(4′-methoxyphenyl)propane-1,3-dione, 4-tert-butyl-4′-methoxy-dibenzoyl methane (Parsol® 1789), 2-(4-diethylamino-2-hydroxybenzoyl)-benzoic acid hexyl ester (Uvinul® A Plus), 1-phenyl-3-(4′-isopropylphenyl)-propane-1,3-dione, as well as enamine compounds. Of course, the UV-A and UV-B filters can also be used in mixtures. Particularly suitable combinations consist of benzoyl methane derivatives such as 4-tert-butyl-4′-methoxydibenzoyl methane (Parsol® 1789) and 2-cyano-3,3-phenylcinnamic acid-2-ethyl-hexyl ester (octocrylene) in combination with esters of cinnamic acid, preferably 4-methoxycinnamic acid-2-ethylhexyl ester and/or 4-methoxycinnamic acid propyl ester and/or 4-methoxycinnannic acid-isoamyl ester. Such combinations have been advantageous combined with water-soluble filters such as 2-phenylbenzimidazole-5-sulfonic acid and alkali, alkaline earth, ammonium, alkylammonium, alkanolammonium, and glucammonium salts thereof.

In a preferred embodiment, if compound (I) is carnosine and the light protection factor is octocrylene, the latter substance should preferably only be present in an amount of less than 5 wt % relative to the composition. If benzophenone-3 is used, the preparation should preferably be free of quinones.

Light Protection Pigments

In addition to the above-mentioned soluble substances, insoluble light protection pigments, specifically finely-dispersed metal oxides or salts, are also suitable for this purpose. Examples of particularly suitable metal oxides are zinc oxide and titanium dioxide, as well as iron, zirconium, silicon, manganese, aluminum, and cerium oxides and mixtures thereof. Silicates (talc), barium sulfate, or zinc stearate can be used as examples of suitable salts. The oxides and salts are used in the form of pigments for skin care and skin protection emulsions and decorative cosmetics. In this case, the particles should have an average diameter of less than 100 nm, preferably 5 to 50 nm, and particularly preferably 15 to 30 nm. They can be spherical in shape, but particles can also be used that are ellipsoid or whose shape is other than spherical The pigments may also be surface-treated, i.e. in a hydrophilized or hydrophobized form. Typical examples are coated titanium dioxides such as titanium dioxide T 805 (Degussa), Eusolex® T2000, Eusolex® T, Eusolex® T-ECO, Eusolex® T-S, Eusolex® T-Aqua, Eusolex® T-45D (all Merck), and Uvinul TiO₂ (BASF). Examples of suitable hydrophobic coating agents in this case are primarily silicones, particularly trialkoxyoctyl silane or simethicone. So-called micro- or nanopigments are preferably used in sun protection agents. Micronized zinc oxides such as Z-COTE® or Z-COTE HP1® are preferably used.

Carriers

Both the drugs and the cosmetic preparations described in the attachment can contain as component (c) carriers or solvents that are selected from the group composed of water, alcohols, esters, butylene glycol, dipropylene glycol, ethanol, ethoxydiglycol, ethyl acetate, glycerol, propanol, isopropanol, macrogols, propyl propylene glycol(2) methyl ether, propyl propylene glycol(3) methyl ether, propylene carbonate, propylene glycol, triethylene glycol, isoparaffin, amyl acetate, amyl benzoate, benzyl acetate, butyl acetate, butylene glycol, butyl lactate, butooctyl benzoate, butooctyl salicylate, C10-C13 alkanes, C14-C17 alkanes, C11-C15 cycloalkanes, caprylyl butyrate, isoparaffins, diacetin, triacetin dicaprylyl ether, dicaprylyl maleate, and mixtures thereof.

Drugs

The drugs according to the invention preferably contain components (a) and (b) in a weight ratio of approx. 1:99 to approx. 99:1, particularly approx. 5:95 to approx. 20:80, and particularly preferably approx. 10:90 to approx. 15:85. The synergistic effect is most pronounced when the two components are used in a weight ratio of approx. 1:5.

A preferred drug has the following composition:

(a) approx. 0.05 to approx. 19.5 wt %, preferably approx. 1 to approx. 15 wt %, and particularly preferably approx. 2 to approx. 8 wt % of a compound of Formula (I), preferably carnosine,
(b) approx. 0.05 to approx. 19.5 wt %, preferably approx. 1 to approx. 15 wt %, and particularly preferably approx. 2 to approx. 8 wt % of at least one UV protection factor and
(c) carriers to 100 wt %.

Cosmetic Preparations

Cosmetic preparations containing the following are also the subject matter of the present invention:

(a) at least one compound of Formula (I)

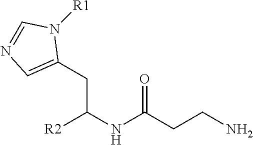

provided that R1 denotes H or CH₃ and R2 denotes H or COOH, or salts thereof, and

(b) at least one UV protection factor, and optionally,

(c1) carriers,

(c2) oil components and/or

(c3) emulsifiers.

The preparations according to the invention are preferably in the form of creams, lotions, gels, pastes, or capsules, and particularly constitute skin care agents, sun protection agents, or hair care agents.

It is further preferred that components (a+b) be present in an amount of 0.1 to 5 wt % relative to the entire composition. In this case, the same preferred weight ratios described above apply.

The cosmetic agents according to the invention can contain other typical auxiliaries and additives, such as mild surfactants, oil components, emulsifiers, pearlizing waxes, consistency-imparting agents, thickeners, superfatting agents, stabilizers, polymers, silicone compounds, fats, waxes, lecithins, phospholipids, moisturizers, biogenic agents, antioxidants, film-forming agents, expanding agents, insect repellents, self-tanning agents, tyrosine inhibitors (depigmenting agents), hydrotropes, solubilizers, preservatives, perfume oils, dyes and the like.

Surfactants

Examples of suitable surface-active substances that may be included are anionic, nonionic, cationic and/or amphoteric or zwitterionic surfactants, ordinarily contained in the agents in amounts of approx. 1 to 70, preferably 5 to 50, and particularly 10 to 30 wt %. Typical examples of anionic surfactants include soaps, alkylbenzene sulfonates, alkane sulfonates, olefin sulfonates, alkyl ether sulfonates, glycerol ether sulfonates, α-methyl ester sulfonates, sulfofatty acids, alkyl sulfates, alkylether sulfates, glycerol ether sulfates, fatty acid ether sulfates, hydroxy mixed ether sulfates, monoglyceride (ether) sulfates, fatty acid amide (ether) sulfates, mono- and dialkylsulfosuccinates, mono- and dialkylsulfosuccinamates, sulfotriglycerides, amide soaps, ether carboxylic acids and salts thereof, fatty acid isethionates, fatty acid sarcosinates, fatty acid taurides, N-acylamino acids such as acyl lactylates, acyl tartrates, acyl glutamates, and acyl aspartates, alkyl oligoglycoside sulfates, protein fatty acid condensates (particularly wheat-based vegetable products) and alkyl(ether) phosphates. If the anionic surfactants contain polyglycol ether chains, they may show a conventional homolog distribution, but preferably a narrow-range homolog distribution. Typical examples of nonionic surfactants are fatty alcohol polyglycol ethers, alkyl phenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkyl glucamides, protein hydrolysates (particularly wheat-based vegetable products), polyol fatty acid esters, sugar esters, sorbitan esters, polysorbates, and amine oxides. If the nonionic surfactants contain polyglycol ether chains, they may show a conventional homolog distribution, but preferably a narrow-range homolog distribution. Typical examples of cationic surfactants are quaternary ammonium compounds such as dimethyl distearyl ammonium chloride, and esterquats, particularly quaternized fatty acid trialkanolamine ester salts. Typical examples of amphoteric or zwitterionic surfactants are alkylbetaines, alkylamidobetaines, aminopropionates, aminoglycinates, imidazolinium betaines, and sulfobetaines. The above-mentioned surfactants are exclusively known compounds. Typical examples of particularly suitable mild surfactants, i.e. those particularly well-tolerated by the skin, are fatty alcohol polyglycol ether sulfates, monoglyceride sulfates, mono- and/or dialkylsulfosuccinates, fatty acid isethionates, fatty acid sarcosinates, fatty acid taurides, fatty acid glutamates, α-olefin sulfonates, ether carboxylic acids, alkyl oligoglycosides, fatty acid glucamides, alkyl amidobetaines, and amphoacetal and/or protein fatty acid condensates, with the latter preferably being based on wheat proteins.

Oil Components

Suitable oil components are, for example, Guerbet alcohols based on fatty alcohols containing 6 to 18, and preferably 8 to 10 carbon atoms, esters of linear C₆-C₂₂ fatty acids with linear or branched C₆-C₂₂ fatty alcohols or esters of branched C₆-C₁₃ carboxylic acids with linear or branched C₆-C₂₂ fatty alcohols, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂ fatty acids with branched alcohols, particularly 2-ethyl hexanol, esters of C₁₈-C₃₈-alkyl hydroxycarboxylic acids with linear or branched C₆-C₂₂ fatty alcohols, particularly dioctyl malate, esters of linear and/or branched fatty acids with polyhydric alcohols (such as propylene glycol, dimer diol, or trimer triol) and/or Guerbet alcohols, triglycerides based on C₆-C₁₀ fatty acids, liquid mono-/di-/triglyceride mixtures based on C₆-C₁₈ fatty acids, esters of C₆-C₂₂ fatty alcohols and/or Guerbet alcohols with aromatic carboxylic acids, particularly benzoic acid, esters of C₂-C₁₂ dicarboxylic acids with linear or branched alcohols with 1 to 22 carbon atoms or polyols with 2 to 10 carbon atoms and 2 to 6 hydroxyl groups, vegetable oils, branched primary alcohols, substituted cyclohexanes, linear and branched C₆-C₂₂ fatty alcohol carbonates such as dicaprylyl carbonate (Cetiol® CC), Guerbet carbonates based on fatty alcohols containing 6 to 18, and preferably 8 to 10 carbon atoms, esters of benzoic acid with linear and/or branched C₆-C₂₂-alcohols (such as Finsolv® TN), linear or branched, symmetrical or asymmetrical dialkyl ethers containing 6 to 22 carbon atoms per alkyl group, such as dicaprylyl ether (Cetiol® OE), ring-opening products of epoxidized fatty acid esters with polyols, silicone oils (cyclomethicone, silicon methicones, etc.) and/or aliphatic or naphthenic hydrocarbons such as squalane, squalene, or dialkyl cyclohexane.

Emulsifiers

Examples of suitable emulsifiers include nonionic surfactants from at least one of the following groups:
addition products of 2 to 30 mol of ethylene oxide and/or 0 to 5 mol of propylene oxide to linear fatty alcohols with 8 to 22 carbon atoms, to fatty acids with 12 to 22 carbon atoms, to alkyl phenols with 8 to 15 carbon atoms in the alkyl group, as well as alkylamines with 8 to 22 carbon atoms in the alkyl residue;
alkyl and/or alkenyl oligoglycosides with 8 to 22 carbon atoms the alk(en)yl residue and ethoxylated analogs thereof;
addition products of 1 to 15 mol of ethylene oxide to castor oil and/or hardened castor oil;
addition products of 15 to 60 mol of ethylene oxide to castor oil and/or hardened castor oil;
partial esters of glycerol and/or sorbitan with unsaturated, linear or saturated, branched fatty acids with 12 to 22 carbon atoms and/or hydroxycarboxylic acids with 3 to 18 carbon atoms, as well as adducts thereof with 1 to 30 mol of ethylene oxide;
partial esters of polyglycerol (average degree of self-condensation 2 to 8), polyethylene glycol (molecular weight 400 to 5000), trimethylolpropane, pentaerythrite, sugar alcohols (such as sorbite), alkyl glycosides (such as methyl glycoside, butyl glycoside, lauryl glycoside), as well as polyglycosides (such as cellulose) with saturated and/or unsaturated, linear or branched fatty acids with 12 to 22 carbon atoms and/or hydroxycarboxylic acids with 3 to 18 carbon atoms, as well as adducts thereof with 1 to 30 mol of ethylene oxide;
mixed esters of pentaerythrite, fatty acids, citric acid and fatty alcohols and/or mixed esters of fatty acids containing 6 to 22 carbon atoms, methyl glucose and polyols, preferably glycerol or polyglycerol.
mono, di- and trialkylphosphates, as well as mono, di- and/or tri-PEG-alkyl phosphates and salts thereof;
wool wax alcohols;
polysiloxane/polyalkyl/polyether copolymers or corresponding derivatives;
block copolymers such as polyethylene glycol-30 dipolyhydroxystearate;
polymer emulsifiers, such as Pemulen polymers (TR-1, TR-2) from Goodrich or Cosmedia® SP from Cognis;
polyalkylene glycols, and
glycerol carbonates.

In the following, particularly suitable emulsifiers are described in further detail:

Alkoxylates.

The addition products of ethylene oxide and/or propylene oxide to fatty alcohols, fatty acids, alkyl phenols, or castor oil constitute known, commercially available products. These are homolog mixtures whose average degree of alkoxylation corresponds to the ratio of the amounts of ethylene oxide and/or propylene oxide to the substrates with the addition reaction was carried out. C_{12/18}-fatty acid mono and diesters of addition products of ethylene oxide to glycerol are known as refatting agents for cosmetic preparations.

Alkyl and/or Alkenyl Oligoglycosides.

Alkyl and/or alkenyl oligoglycosides and the production and use thereof are known from prior art. In particular they are produced by reacting glucose or oligosaccharides with primary alcohols having 8 to 18 carbon atoms. With the respect to the glycoside residue, both monoglycosides, in which a cyclic sugar residue is glycosidically bonded to the fatty alcohol, and oligomeric glycosides, preferably having a degree of oligomerization of approx. 8, are suitable. In this case, the degree of oligomerization is an average statistical value based on the usual homolog distribution for such technical products.

Partial Glycerides.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric diglyceride, malic acid monoglyceride, malic acid diglyceride, and technical mixtures thereof that can secondarily contain small amounts of triglycerides from the production process. Addition products of 1 to 30, and preferably 5 to 10 mol of ethylene oxide to the above-mentioned partial glycerides are also suitable.

Sorbitan Esters.

Examples of suitable sorbitan esters include sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical mixtures thereof. Addition products of 1 to 30 and preferably 5 to 10 mol of ethylene oxide to the above-mentioned sorbitan esters are also suitable.

Polyglycerol Esters.

Typical examples of suitable polyglycerol esters are polyglyceryl-2 dipolyhydroxystearate (Dehymuls® PGPH), polyglyceryl-3-diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® NL), polyglyceryl-3 distearate (Cremophor® GS 32), polyglyceryl polyricinoleate (Admul® WOL 1403), polyglyceryl dimerate isostearate, and mixtures thereof. Examples of further suitable polyol esters are mono, di, and triesters, optionally reacted with 1 to 30 mol of ethylene oxide, of trimethylol propane or pentaerythrite with lauric acid, coconut fatty acid, tallow fatty acid, palmitic acid, stearic acid, oleic acid, behenic acid and the like.

Anionic Emulsifiers.

Typical anionic emulsifiers are aliphatic fatty acids with 12 to 22 carbon atoms, such as palmitic acid, stearic acid or behenic acid, as well as dicarboxylic acids with 12 to 22 carbon atoms, such as azelaic acid or sebacic acid.

Amphoteric and Cationic Emulsifiers.

Zwitterionic surfactants can also be used as emulsifiers. Zwitterionic surfactants are surface-active compounds that carry at least one quaternary ammonium group and at least one carboxylate and a sulfonate group in the molecule. Particularly suitable zwitterionic surfactants are the so-called betaines, including N-alkyl-N,N-dimethylammonium glycinates such as coconut alkyl dimethylammonium glycinate, N-acylaminopropyl-N,N-dimethylammonium glycinates such as coconut acylaminopropyldimethyl ammoniumglycinate, and 2-alkyl-3-carboxylmethyl-3-hydroxyethylimidazolines containing 8 to 18 carbon atoms in their alkyl or acyl groups, as well as coconut acylaminoethyl hydroxyethyl carboxymethyl glycinate. Particularly preferred is the fatty acid amide derivative known under the CTFA name cocamidopropyl betaine. Ampholytic surfactants are also suitable emulsifiers. Ampholytic surfactants are surface-active compounds that, in addition to a C_{8/18}alkyl or acyl group, contain at least one free amino group and at least one —COOH— or —SO₃H group in the molecule and are capable of forming inner salts. Examples of suitable ampholytic surfactants include N-alkyl glycines, N-alkyl propionic acids, N-alkyl aminobutyric acids, N-alkyl iminodipropionic acids, N-hydroxyethyl-N-alkyl amidopropylglycine, N-alkyl taurine, N-alkyl sarcosine, 2-alkyl aminopropionic acids and alkyl aminoacetic acids with approx. 8 to 18 carbon atoms in their alkyl groups. Particularly preferred ampholytic surfactants are N-coconut alkyl aminopropionate, coconut acyl aminoethylaminopropionate, and C_{12/18} acyl sarcosine. Finally, cationic surfactants are also suitable as emulsifiers, with those of the esterquat type, preferably methyl quaternized difatty acid triethanolamine ester salts, being particularly preferred.

Fats and Waxes

Typical examples of fats are glycerides, i.e. solid or liquid vegetable or animal products consisting essentially of mixed glycerol esters of higher fatty acids; examples of suitable waxes include natural waxes, such as candelilla wax, carnauba wax, Japan wax, esparto grass wax, cork wax, guaruma wax, rice germ oil wax, sugar cane wax, ouricury wax, montan wax, beeswax, shellac wax, spermaceti, lanolin (wool wax), uropygial fat, ceresin, ozocerite (earth wax), petrolatum, paraffin waxes, and microwaxes; chemically modified waxes (hard waxes), such as montan ester waxes, sasol waxes, hydrogenated jojoba waxes, as well as synthetic waxes such as polyalkylene waxes and polyethylene glycol waxes. In addition to the fats, fat-like substances such as lecithins and phospholipids are also suitable as additives. The person skilled in the art understands the term lecithins to refer to glycero-phospholipids formed from fatty acids, glycerol, phosphoric acid, and choline by esterification. Lecithins are therefore frequently referred to by specialists as phosphatidyl cholines (PC). Examples of suitable natural lecithins include the kephalins, also referred to as phosphatidic acids, and which are derivatives of 1,2-diacyl-sn-glycerol-3-phosphoric acids. In contrast, phospholipids are ordinarily understood to be mono- and preferably diesters of phosphoric acid with glycerol (glycerol phosphates) that are generally classified as fats. In addition, sphingosines or sphingolipids are also suitable.

Examples of suitable pearlizing waxes include alkylene glycol esters, particularly ethylene glycol distearate; fatty acid alkanolamides, particularly coconut fatty acid diethanolamide; partial glycerides, particularly stearic acid monoglyceride; esters of polyvalent, optionally hydroxy-substituted carboxylic acids with fatty alcohols containing 6 to 22 carbon atoms, particularly long-chain esters of tartaric acid; fatty substances such as fatty alcohols, fatty ketones, fatty aldehydes, fatty ethers, and fatty carbonates that have a total of at least 24 carbon atoms, particularly laurone and distearyl ether; fatty acids such as stearic acid, hydroxystearic acid, or behenic acid, ring opening products of olefin epoxides having 12 to 22 carbon atoms with fatty alcohols having 12 to 22 carbon atoms and/or polyols having 2 to 15 carbon atoms and 2 to 10 hydroxyl groups, as well as mixtures thereof.

Coolants

Coolants are compounds that produce a feeling of coolness on the skin. As a rule, these are menthol compounds, which—in addition to the base component menthol itself—contain substances selected from the group comprising menthol methyl ether, menthone glyceryl acetal (FEMA GRAS¹ 3807), menthone glyceryl ketal (FEMA GRAS 3808), menthyl lactate (FEMA GRAS 3748), menthol ethylene glycol carbonate (FEMA GRAS 3805), menthol propylene glycol carbonate (FEMA GRAS 3806), menthyl-N-ethyloxamate, monomethyl succinate (FEMA GRAS 3810), monomenthyl glutamate (FEMA GRAS 4006), menthoxy-1,2-propane diol (FEMA GRAS 3784), menthoxy-2-methyl-1,2-propane diol (FEMA GRAS 3849), and the methane carboxylic acid esters and amides WS-3, WS-4, WS-5, WS-12, WS-14, and WS30, as well as mixtures thereof. FEMA stands for “Flavor and Extracts Manufacturers Association,” and GRAS is defined as “Generally Regarded As Safe.” A FEMA GRAS designation means that the substance identified in this manner has been tested using standard methods and is assessed to be toxicologically unobjectionable.

A first important representative of these substances is monomenthyl succinate (FEMA GRAS 3810). Both the succinate and the analogous monomenthyl glutarate (FEMA GRAS 4006) constitute important representatives of monomenthyl esters based on di- and polycarboxylic acids:

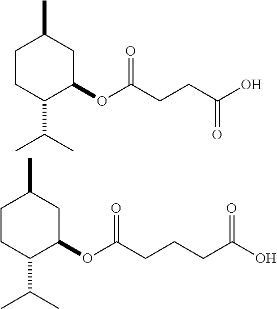

Examples of uses of these substances can be found for example in the documents WO 2003 043431 (Unilever) or EP 1332772 A1 (IFF).

The next important group of preferred menthol compounds within the meaning of the invention comprises carbonate esters of menthol and polyols, including glycols, glycerol, or carbohydrates, such as menthol ethylene glycol carbonate (FEMA GRAS 3805=Frescolat® MGC), menthol propylene glycol carbonate (FEMA GRAS 3784=Frescolat® MPC), menthol 2-methyl-1,2-propane diol carbonate (FEMA GRAS 3849) or the corresponding sugar derivatives. Also preferred are the menthol compounds menthyl lactate (FEMA GRAS 3748=Frescolat® ML), and particularly menthone glyceryl acetal (FEMA GRAS 3807) or menthone glyceryl ketal (FEMA GRAS 3808), which is marketed under the name Frescolat® MGA. Among these substances, menthone glyceryl acetal/ketal, menthyl lactate, menthol ethylene glycol carbonate, or menthol propylene glycol carbonate have been found to be particularly advantageous, and are marketed by the Applicant under the names Frescolat® MGA, Frescolat® ML, Frescolat® MGC, and Frescolat® MPC.

Menthol compounds having a C—C bond at position 3 and from which a series of representatives can also be used were first developed in the 1970s. These substances are generally referred to as WS types. The base component is a menthol derivative in which the hydroxyl group has been replaced with a carboxyl group (WS-1). All other types of WS, such as the preferred species WS-3, WS-4, WS-5, WS-12, WS-14 and WS-30, are derived from this structure.

Consistency-Imparting Agents and Thickeners

Suitable consistency-imparting agents are primarily fatty alcohols or hydroxy fatty alcohols with 12 to 22, and preferably 16 to 18 carbon atoms, as well as partial glycerides, fatty acids, or hydroxy fatty acids. A combination of these substances with alkyl oligoglycosides and/or fatty acid-N-methylglucamides of the same chain length and/or polyglyceryl poly-12-hydroxystearates is preferred. Examples of suitable thickeners are aerosil types (hydrophilic silicic acids), polysaccharides, particularly xanthan gum, guar-guar, agar-agar, alginates and tyloses, carboxymethylcellulose and hydroxyethyl- and hydroxypropylcellulose, as well as higher-molecular polyethylene glycol mono- and diesters of fatty acids, polyacrylates (such as Carbopole® and Pemulen products from Goodrich; Synthalene® from Sigma; Keltrol products from Kelco; Sepigel products from Seppic; Salcare products from Allied Colloids) polyacrylamides, polymers, polyvinyl alcohol, and polyvinyl pyrrolidone. Bentonites such as Bentone® Gel VS-5PC (Rheox) have also been found to be particularly effective, comprising a mixture of cyclopentasiloxane, disteardimonium hectorite, and propylene carbonate. Also suitable are surfactants such as ethoxylated fatty acid glycerides, esters of fatty acids with polyols such as pentaerythrite or trimethylol propane, fatty alcohol ethoxylates having narrow-range homolog distribution, or alkyl oligoglycosides, as well as electrolytes such as table salt and ammonium chloride.

Superfatting Agents and Stabilizers

Examples of suitable superfatting agents are substances such as lanolin and lecithin, as well as polyethoxylated or acylated lanolin and lecithin derivatives, polyol fatty acid esters, monoglycerides, and fatty acid alkanolamides, wherein the latter simultaneously serve as foam stabilizers.

Metal salts of fatty acids, such as magnesium, aluminum and/or zinc stearate or ricinoleate, can be used as stabilizers.

Polymers

Examples of suitable cationic polymers include cationic cellulose derivatives such as a quaternized hydroxyethylcellulose available under the name Polymer JR 400® from Amerchol, cationic starch, copolymers of diallyl ammonium salts and acrylamides, quaternized vinyl pyrrolidone/vinyl imidazole polymers such as Luviquat® (BASF), condensation products of polyglycols and amines, quaternized collagen polypeptides such as lauryldimonium hydroxypropyl hydrolyzed collagen (Lamequat®L/Grünau), quaternized wheat polypeptides, polyethylene imine, cationic silicone polymers such as amodimethicone, copolymers of adipic acid and dimethylaminohydroxypropyldiethylene triamine (Cartaretine®/Sandoz), copolymers of acryl acid with dimethyl diallyl ammonium chloride (Merquat® 550/Chemviron), polyaminopolyamides and crosslinked water-soluble polymers thereof, cationic chitin derivatives such as quaternized chitosan, optionally distributed in microcrystalline form, condensation products of dihalogen alkylene such as dibromobutane with bis-dialkylamines such as bis-dimethylamino-1,3-propane, cationic guar-gums such as Jaguar® CBS, Jaguar® C-17, and Jaguar® C-16 from Celanese, and quaternized ammonium salt polymers such as Mirapol® A-15, Mirapol® AD-1, and Mirapol® AZ-1 from Miranol.

Examples of suitable anionic, zwitterionic, amphoteric, and nonionic polymers include vinyl acetate/crotonic acid copolymers, vinyl pyrrolidone/vinyl acrylate copolymers, vinyl acetate/butyl maleate/isobornyl acrylate copolymers, methylvinyl ether/maleic acid anhydride copolymers and esters thereof, non-crosslinked polyacrylic acids and polyacrylic acids crosslinked with polyols, acrylamidopropyl trimethylammonium chloride/acrylate copolymers, octylacrylamide/methyl methacrylate/tert-butyl aminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers, and optionally, derivatized cellulose ethers and silicones.

Silicone Compounds

Suitable silicone compounds are for example dimethyl polysiloxane, methylphenyl polysiloxane, cyclic silicones, as well as amino, fatty acid, alcohol, polyether, epoxy, fluorine, glycoside, and/or alkyl-modified silicone compounds, which can be present at room temperature either in liquid or resinous form. Also suitable are simethicones, which are mixtures of dimethicones having an average chain length of 200 to 300 dimethylsiloxane units and hydrogenated silicates.

Moisturizers

Moisturizers are used for further optimization of the sensory properties of the composition and for moisture regulation of the skin. At the same time, the cold stability of the preparations according to the invention is increased, particularly in the case of emulsions. The moisturizers are ordinarily contained in an amount of 0.1 to 15 wt %, preferably 1 to 10 wt %, and particularly preferably 5 to 10 wt %.

Examples of suitable moisturizers according to the invention include amino acids, pyrrolidone carboxylic acid, lactic acid and salts thereof, lactitol, urea and urea derivatives, uric acid, glucosamine, creatinine, cleavage products of collagen, chitosan or chitosan salt derivatives, and particularly polyols and polyol derivatives (such as glycerol, diglycerol, triglycerol, ethylene glycol, propylene glycol, butylene glycol, erythrite, 1,2,6-hexane triol, polyethylene glycols such as PEG-4, PEG-6, PEG-7, PEG-8, PEG-9, PEG-10, PEG-12, PEG-14, PEG-16, PEG-18, and PEG-20), sugar and sugar derivatives (including fructose, glucose, maltose, maltitol, mannite, inosite, sorbite, sorbityl silane diol, sucrose, trehalose, xylose, xylite, glucuronic acid and salts thereof), ethoxylated sorbite (sorbeth-6, sorbeth-20, sorbeth-30, sorbeth-40), honey and hardened honey, hardened starch hydrolysates, as well as mixtures of hardened wheat protein and PEG-20/acetate copolymer. Preferred suitable moisturizers according to the invention are glycerol, diglycerol, triglycerol, and butylene glycol.

Biogenic Active Ingredients and Antioxidants

Biogenic active ingredients are understood to be e.g. tocopherol, tocopherol acetate, tocopherol palmitate, ascorbic acid, (deoxy)ribonucleic acid and fragmentation products thereof, β-glucan, retinol, bisabolol, allantoin, phytantriol, panthenol, AHA acids, amino acids, ceramides, pseudoceramides, essential oils, and plant extracts such as prune extract, bambara extract, and vitamin complexes.

Antioxidants interrupt the photochemical reaction chain triggered when UV radiation penetrates the skin. Typical examples of these are amino acids (such as glycine, histidine, tyrosine, tryptophan) and derivatives thereof, imidazoles (such as urocanic acid) and derivatives thereof, peptides such as D,L-carnosine, D-carnosine, L-carnosine and derivatives thereof (such as anserine), carotenoids, carotenes (such as α-carotene, β-carotene, lycopene) and derivatives thereof, chlorogenic acid and derivatives thereof, liponic acid and derivatives thereof (such as dihydroliponic acid), aurothioglucose, propylthiouracil and other thiols (such as thioredoxin, glutathione, cysteine, cystine, cystamine and glycosyls thereof, N-acetyl, methyl, ethyl, propyl, amyl, butyl and lauryl, palmitoyl, oleyl, γ-linoleyl, cholesteryl and glyceryl esters), as well as salts thereof, dilauryl thiodipropionate, distearyl thiodipropionate, thiodipropionic acid and derivatives thereof (esters, ethers, peptides, lipids, nucleotides, nucleosides and salts), as well as sulfoximine compounds (such as buthionine sulfoximine, homocysteine sulfoximine, buthionine sulfone, penta, hexa, heptathionine sulfoximine) in very low tolerated doses (such as pmol to μmol/kg), as well as (metal) chelators (such as α-hydroxy fatty acids, palmitic acid, phytic acid, and lactoferrin), α-hydroxy acids (such as citric acid, lactic acid, and malic acid), humic acid, gallic acid, gall extracts, bilirubin, biliverdin, EDTA, EGTA and derivatives thereof, unsaturated fatty acids and derivatives thereof (such as γ-linolenic acid, linoleic acid, and oleic acid), folic acid and derivatives thereof, ubiquinone, ubiquinol and derivatives thereof, Vitamin C and derivatives thereof (such as ascorbyl palmitate, Mg ascorbyl phosphate, ascorbyl acetate), tocopherols and derivatives thereof (such as Vitamin E acetate), Vitamin A and derivatives thereof (vitamin A palmitate), as well as coniferyl benzoate of benzoin, rutic acid and derivatives thereof, α-glycosylrutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (such as ZnO, ZnSO₄), selenium and derivatives thereof (such as selenium methionine), stilbene and derivatives thereof (such as stilbene oxide, trans-stilbene oxide), and suitable derivatives of the above-mentioned active ingredients according to the invention (salts, esters, ethers, sugars, nucleotides, nucleosides, peptides, and lipids).

Film-Forming Agents, Antidandruff Agents, and Expanding Agents

Examples of common film-forming agents include chitosan, microcrystalline chitosan, quaternized chitosan, polyvinyl pyrrolidone, vinyl pyrrolidone-vinyl acetate copolymerisates, polymers of the acrylic acid series, quaternary cellulose derivatives, collagen, hyaluronic acid or salts thereof, and similar compounds.

Examples of suitable antidandruff active ingredients include piroctone olamine (1-hydroxy-4-methyl-6-(2,4,4-trimethylpentyl)-2-(1H)-pyridinone monoethanolamine salt), Baypival® (climbazole), Ketoconazol®, (4-acetyl-1-{-4-[2-(2,4-dichlorophenyl) r-2-(1H-imidazol-1-ylmethyl)-1,3-dioxylan-c-4-ylmethoxyphenyl}piperazine, ketoconazole, Elubiol, selenium disulfide, colloidal sulfur, sulfur polyethylene glycol sorbitan monooleate, sulfur rizinol polyethoxylate, sulfur-tar distillates, salicylic acid (or in combination with hexachlorophene), undecylenic acid monoethanolamide sulfosuccinate Na salt, Lamepon® UD (protein-undecylenic acid condensate), zinc pyrithione, aluminum pyrithione, and magnesium pyrithione/dipyrithione magnesium sulfate.

Examples of suitable expanding agents for aqueous phases are montmorillonite, clay mineral substances, Pemulen, as well as alkyl-modified carbopol products (Goodrich). Further suitable polymers or expanding agents can be seen in the overview of R. Lochhead in Cosm. Toil. 108, 95 (1993).

Insect Repellents

Examples of suitable insect repellents include N,N-diethyl-m-toluamide, 1,2-pentane diol, or ethyl butyl acetyl aminopropionates. Suitable self-tanning agents include dihydroxyacetone. Examples of suitable tyrosine inhibitors, which prevent the formation of melanin and are used in depigmentation agents, include arbutin, ferulic acid, kojic acid, cumaric acid, and ascorbic acid (Vitamin C).

Hydrotropes

Moreover, hydrotropes, such as ethanol, isopropyl alcohol, or polyols can be used in order to improve flow properties; these substances largely correspond to the carriers described at the outset. In this case, suitable polyols preferably have 2 to 15 carbon atoms, and at least two hydroxyl groups. The polyols can also include other functional groups, particularly amino groups, or be modified with nitrogen. Typical examples are
glycerol;
alkylene glycols, such as ethylene glycol, diethylene glycol, propylene glycol, butylene glycol, hexylene glycol, as well as polyethylene glycols with an average molecular weight of 100 to 1,000 daltons;
technical oligoglycerol mixtures having a degree of self-condensation of 1.5 to 10 such as technical diglycerol mixtures with a diglycerol content of 40 to 50 wt %;
methylol compounds, particularly trimethylol ethane, trimethylol propane, trimethylol butane, pentaerythrite, and dipentaerythrite;
lower alkyl glycosides, particularly those with 1 to 8 carbon atoms in the alkyl residue, such as methyl and butyl glycoside;
sugar alcohols with 5 to 12 carbon atoms, such as sorbite or mannite,
sugars with 5 to 12 carbon atoms, such as glucose or saccharose;
amino sugars, such as glucamine;
dialcoholamines, such as diethanolamine or 2-amino-1,3-propane diol.

Preservatives

Examples of suitable preservatives include phenoxyethanol, formaldehyde solution, parabens, pentane diol, or sorbic acid, as well as the silver complexes known under the name Surfacine® and the additional substance classes listed in Appendix 6, sections A and B of the Cosmetics Ordinance.

Perfume Oils and Fragrances

Examples of suitable perfume oils include mixtures of natural and synthetic fragrances. Natural fragrances are flower extracts (lily, lavender, rose, jasmine, neroli, ylangylang), stems and leaves (geranium, patchouli, petitgrain), fruits (anise, coriander, caraway, juniper), fruit peels (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials such as civet and beaver may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert-butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include benzyl ethyl ether, while aldehydes include linear alkanals containing 8 to 18 carbon atoms, citral, citronellal, citronellyl oxyacetaldehyde, cyclamen aldehyde, hydroxycitronellal, lilial and bourgeonal; examples of suitable ketones are the ionones, aisomethylionone, and methyl cedryl ketone. Suitable alcohols are anethol, citronellol, eugenol, isoeugenol, geraniol, linalool, phenylethyl alcohol, and terpineol. The hydrocarbons chiefly include the terpenes and balsams. However, mixtures of different perfume compounds are preferred that produce an agreeable fragrance together. Other suitable perfume oils include essential oils of low volatility that are mostly used as aroma components, such as sage oil, camomile oil, clove oil, melissa oil, mint oil, cinnamon leaf oil, lime-blossom oil, juniper berry oil, vetiver oil, olibanum oil, galbanum oil, ladanum oil, and lavendin oil. Preferably, bergamot oil, dihydromyrcenol, lilial, lyral, citronellol, phenylethyl alcohol, α-hexylcinnamaldehyde, geraniol, benzyl acetone, cyclamen aldehyde, linalool, boisambrene forte, ambroxan, indole, hedione, sandelice, citrus oil, mandarin oil, orange oil, allyl amyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillate, irotyl, and floramate are used either individually or in mixtures.

Examples of suitable fragrances include peppermint oil, spearmint oil, anise oil, star anise oil, caraway oil, eucalyptus oil, fennel oil, citrus oil, wintergreen oil, clove oil, menthol, and the like.

Dyes

The dyes that can be used are those suitable and approved for cosmetic purposes, such as those listed in the publication “Cosmetic Dyes” of the Farbstoffkommission der Deutschen Forschungsgemeinschaft [Dyes Commission of the German Research Foundation], Verlag Chemie, Weinheim, 1984, pp. 81-106. Examples are cochineal red A (C.I. 16255), patent blue V (C.I.42051), indigotin (C.I.73015), chlorophyllin (C.I.75810), quinoline yellow (C.I.47005), titanium dioxide (C.I.77891), indanthrene blue RS (C.I. 69800) and alizarin red (C.I.58000). Luminol can also be included as a luminescent dye. These dyes are ordinarily used in concentrations of 0.001 to 0.1 wt % relative to the entire mixture.

The total amount of these auxiliaries and additives can be 1 to 50, and preferably 5 to 40 wt % relative to the agent. The agent can be produced by common cold or hot processes; the phase inversion temperature mode is preferred.

### INDUSTRIAL APPLICABILITY

The use, particularly the non-therapeutic use, of preparations containing the following is also the subject matter of the invention:
(a) at least one compound of Formula (I)

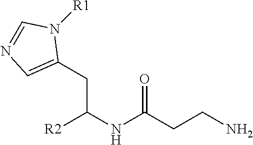

provided that R1 denotes H or CH₃ and R2 denotes H or COOH, or salts thereof, and
(b) at least one UV protection factor
   for protecting the skin against UV radiation.

In this case, components (a) and (b) are preferably used in a weight ratio of approx. 1:99 to approx. 99:1, particularly preferably approx. 5:95 to approx. 20:80, and most particularly preferably approx. 10:90 to approx. 15:85. Moreover, the above-described preferred embodiments also apply mutatis mutandis to use of the substances.

### EXAMPLES

### Examples 1 to 5, Comparative Examples V1 to V4

Inhibition of MMP-1 Synthesis

The capacity of the test substances to alleviate the toxic effect of UVA radiation was investigated. A culture of dermal fibroblasts was used as an in vitro system, and the release of MMP-1 from these fibroblasts as an effect of UV radiation was determined. In order to carry out the experiment, a fibroblast culture with fetal calf serum was prepared and inoculated with the test substances 2 days thereafter. After incubation for 36 h at 37° C. with a CO₂ level of 5 vol %, the nutrient medium was replaced with an electrolyte solution, and the fibroblasts were damaged with a specified dose of UVB radiation (50 mJ/cm²). Determination of MMP was carried out with a kit that is commercially available from the firm Amersham under the name RPN2610. The results are shown in Table 1. The amount of MMP is given in ng/ml from a test series with threefold determination.

TABLE 1
MMP inhibition (in rel. %)

| | | Conc. | MMP | |
|---|---|---|---|---|
| | | % | without | |
| Exp. | Test products | w/v | UVA | with UVA |
| | Control | — | 49 ± 9 | 199 ± 25 |
| V1 | L-carnosine | 0.001 | 22 ± 5 | 101 ± 17 |
| V2 | Carnitine*HCl | 0.001 | 24 ± 7 | 118 ± 21 |
| V3 | Uvinul A | 0.001 | 17 ± 3 | 99 ± 12 |
| V4 | Octocrinyl | 0.001 | 17 ± 7 | 100 ± 20 |
| 1 | L-carnosine + Uvinul A (1:1) | 0.001 | 12 ± 4 | 88 ± 12 |
| 2 | L-carnosine + octocrinyl (1:1) | 0.001 | 14 ± 3 | 92 ± 15 |
| 3 | L-carnosine + Uvasorb HEB (1:5) | 0.001 | 13 ± 1 | 91 ± 9 |
| 4 | L-carnosine + NeoHeliopan (1:5) | 0.001 | 14 ± 5 | 92 ± 7 |
| 5 | L-carnosine + titanium dioxide (1:10) | 0.001 | 16 ± 7 | 98 ± 11 |

The results show that the test substances inhibit the release of MMP in UVA irradiation in a lasting manner and have a synergistic effect compared to the individual substances.

### Application Examples

The following examples show formulations for various sun protection products that contain the preparations according to the invention. All figures are to be understood as indicating wt %.

Cosmetic Sun Protection Agent

| Components | Amount |
|---|---|
| Ethylhexyl cinnamic acid | 7.50 |
| Benzophenone-3 | 2.00 |
| Polyglyceryl dimer soyate | 0.80 |
| Sorbitan stearate | 1.00 |
| Tocopheryl acetate | 0.50 |
| Glyceryl stearate. PEG-100 Stearate | 3.00 |
| PEG-40. Hydrogenated castor oil | 1.00 |
| Titanium dioxide. Aluminum oxide hydrate. Dimethicone/ | 3.00 |
| methicone copolymer | |
| Butyrospermum parkii (shea butter) | 1.00 |
| C₁₂₋₁₅ alkyl benzoate | 6.50 |
| Butylene glycol | 5.00 |
| Xanthan gum | 0.30 |
| Disodium EDTA | 0.10 |
| Allantoin | 0.10 |
| Polyacrylamide. C₁₃₋₁₄ isoparaffin. Laureth-7 | 1.00 |
| Pentylene glycol | 5.00 |
| 4-t-Butyl cyclohexanol | 1.00 |
| Carnosine | 0.30 |
| Preservatives (methyl, butyl, ethyl, propylparaben, | 0.30 |
| phenoxyethanol) | |
| Substance of formula (I) | 0.60 |
| Aqua dem. | Ad 100 |

Sun Protection Spray

| Components | INCI | Amount |
|---|---|---|
| Water, demineralized | Water (aqua) | 69.50 |
| Glycerol | Glycerol | 4.00 |
| 1,3-butylene glycol | Butylene glycol | 5.00 |
| D-Panthenol | Panthenol | 0.50 |
| Lara care A-200 | Galactoarabinan | 0.25 |
| Baysilone oil M 10 | Dimethicone | 1.00 |
| Edeta BD | Disodium EDTA | 0.10 |
| Copherol 1250 | Tocopheryl acetate | 0.50 |
| Cetiol OE | Dicaprylyl ether | 3.00 |
| Neo Heliopan ® HMS | Homosalate | 5.00 |
| Neo Heliopan ® AV | Ethylhexyl methoxycinnamate | 6.00 |
| Neo Heliopan ® 357 | Butyl methoxydibenzoyl methane | 1.00 |
| Corapan TQ | Diethylhexylnaphthalate | 2.00 |
| Alpha Bisabolol | Bisabolol | 0.10 |
| Pemulen TR-2 | Acrylates/C₁₀₋₃₀ alkyl | 0.25 |
| | acrylate crosspolymer | |
| NaOH 10% | Sodium hydroxide | 0.60 |
| Perfume oil | Fragrance | 0.20 |
| Phenoxyethanol | Phenoxyethanol | 0.40 |
| Solbrol M | Methylparaben | 0.10 |
| Solbrol P | Propylparaben | 0.10 |
| Carnosine | Carnosine | 0.50 |

Sun Protection Spray OM SPF 15-20

| Components | INCI | Amount |
|---|---|---|
| Dracorin ® GOC | Glyceryl oleate citrate. | 2.00 |
| | Caprylic/capric | |
| | triglyceride | |
| Corapan ® TQ | Diethylhexyl 2,6- | 3.00 |
| | naphthalate | |
| Neo Heliopan ® HMS | Homosalate | 7.00 |
| Neo Heliopan ® OS | Ethylhexyl salicylate | 5.00 |
| Neo Heliopan ® 357 | Butyl methoxydibenzoyl | 3.00 |
| | methane | |
| Isoadipate | Diisopropyl adipate | 6.00 |
| Baysilone ® Oil M10 | Dimethicone | 1.00 |
| Edeta ® BD | Disodium EDTA | 0.10 |
| Vitamin E acetate | Tocopheryl acetate | 0.50 |
| Dragosantol ® 100 | Bisabolol | 0.10 |
| Pemulen ® TR-2 | Acrylates/C₁₀₋₃₀ alkyl | 0.25 |
| | acrylate crosspolymer | |
| Water | Water (Aqua) | Ad 100 |
| Glycerol 99.5 P | Glycerol | 4.00 |
| Butylene glycol | Butylene glycol | 5.00 |
| Neo Heliopan ® hydro (103089). | Phenylbenzimidazole | 8.00 |
| Used as 25% aq. solution | sulfonic acid | |
| neutralized with Biotive ® | | |
| L-Arginine | | |
| Biotive ® L-Arginine | Arginine | 0.55 |
| Perfume oil | Fragrance | 0.40 |
| Sobrol M | Methylparaben | 0.30 |
| Carnosine | Carnosine | 0.60 |

Sun Protection Soft Cream W/O SPF 40

| Components | INCI | Amount |
|---|---|---|
| Dehymuls PGPH | Polyglyceryl-2 | 5.00 |
| | dipolyhydroxystearate | |
| Copherol 1250 | Tocopheryl acetate | 0.50 |
| Permulgin 3220 | Ozocerite | 0.50 |
| Zinc stearate | Zinc stearate | 0.50 |
| Tegosoft TN | C₁₂₋₁₅ alkyl benzoate | 10.00 |
| Neo Heliopan ® E1000 | Isoamyl-p-methoxycinnamate | 2.00 |
| Neo Heliopan ® 303 | Octocrylene | 5.00 |
| Neo Heliopan ® MBC | 4-methylbenzylidene camphor | 3.00 |
| Zinc oxide, neutral | Zinc oxide | 5.00 |
| Water, distilled | Water (aqua) | Add 100 |
| EDETA BD | Disodium EDTA | 0.10 |
| Glycerol | Glycerol | 4.00 |
| Magnesium sulfate | Magnesium sulfate | 0.50 |
| Perfume oil P1, P2, P3, or P4 | Perfume | 0.30 |
| Symdiol ® 68 | 1,2-hexane diol. Caprylyl | 0.30 |
| | glycol | |
| Carnosine | Carnosine | 0.80 |

Sun Protection Lotion W/O

| Components | INCI | Amount |
|---|---|---|
| Dehymuls PGPH | Polyglyceryl-2 | 3.00 |
| | dipolyhydroxystearate | |
| Beeswax 8100 | Beeswax | 1.00 |
| Monomuls 90-0-18 | Glyceryl oleate | 1.00 |
| Zinc stearate | Zinc stearate | 1.00 |
| Cetiol SN | Cetearyl isononanoate | 5.00 |
| Cetiol OE | Dicaprylyl ether | 5.00 |
| Tegosoft TN | C12-15 alkyl benzoate | 4.00 |
| Vitamin E | Tocopherol | 0.50 |
| Neo Heliopan ® OS | Ethylhexyl salicylate | 5.00 |
| Neo Heliopan ® AV | Ethylhexyl methoxycinnamate | 7.50 |
| Uvinul ® T150 | Ethylhexyl triazone | 1.50 |
| Water, distilled | Water (Aqua) | ad 100 |
| Trilon BD | Disodium EDTA | 0.10 |
| Glycerol | Glycerol | 5.00 |
| Neo Heliopan ® AP 10% | Disodium phenyl | 15.00 |
| solution. Neutralized | dibenzimidazole | |
| with NaOH | tetrasulfonate | |
| Perfume oil | Perfume | 0.25 |
| Alpha bisabolol | Bisabolol | 0.10 |
| SymOcide ® PT | Phenoxyethanol. Tropolone | 0.25 |
| Carnosine | Carnosine | 0.25 |

After-Sun Gel

| Components | INCI | Amount |
|---|---|---|
| SymSol ® PF-3 | Water (aqua). Pentylene glycol. Sodium | 3.00 |
| | lauryl sulfoacetate. Sodium oleoyl | |
| | sarcosinate. Sodium chloride. Disodium | |
| | sulfoacetate. Sodium oleate. Sodium | |
| | sulfate | |
| Glycerol 99.5 P. | Glycerol | 5.00 |
| SymHelios ® 1031 | Benzylidene dimethoxy dimethylene | 0.10 |
| | danone | |
| Water | Water (aqua) | Ad 100 |
| Pemulen ® TR-2 | Acrylates/C₁₀₋₃₀ alkyl | 1.00 |
| | acrylate crosspolymer | |
| D-Panthenol 75 W | Panthenol | 0.50 |
| SymFinity ® 1298 | Echinacea purpurea extract | 0.10 |
| Extrapone ® | Water (aqua). Glycerol. Hydrolyzed | 1.00 |
| Pearl GW | pearl. Xanthan gum | |
| Sodium hydroxide | Sodium hydroxide | 2.50 |
| 10% solution | | |
| Ethanol 96% | Alcohol denat. | 15.00 |
| Perfume oil | Perfume | 0.20 |
| SymOcide ® PS | Phenoxyethanol. 1,2-Hexanediol. | 0.50 |
| | Decylene glycol | |
| Carnosine | Carnosine | 0.10 |

1-15. (canceled)

16. A method for the inhibition of MMP-1 expression in human skin induced by UV radiation, comprising the steps of:
(i) providing a composition comprising
(a) at least one compound of the following Formula (I) 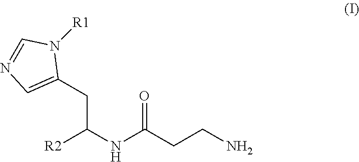
provided R1 denotes H or CH₃ and R2 denotes or COOH or salts thereof,
(b) at least one UV protection factor, and
(c) a carrier, and
(ii) applying said composition onto human skin.

17. The method of claim 16, wherein the compound of Formula (I) is selected from the group consisting of carnosine, L-carnosine, D-carnosine, D/L-carnosine, carnitine, carnitine*HCl, anserine, D-anserine, L-anserine, L-anserine*HNO₃ and mixtures thereof.

18. The method of claim 16, wherein the UV protection factor is selected from the group consisting of UV-A filters and UV-B filters and mixtures thereof.

19. The method of claim 18, wherein the UV protection factor is selected from the group consisting of 3-benzylidene camphor or 3-benzylidene norcamphor and derivatives thereof, 4-aminobenzoic acid derivatives, esters of cinnamic acid, esters of salicylic acid, benzophenone derivatives, esters of benzylmalonic acid, triazine derivatives, propane-1,3-diones, ketotricyclo(5.2.1.0)decane-derivatives, 2-phenylbenzimidazole-5-sulfonic acid, and salts thereof; sulfonic acid derivatives of benzophenones, sulfonic acid derivatives of 3-benzylidene camphor, and mixtures thereof.

20. The method of claim 18, wherein the UV protection factor is selected from the group consisting of diethylamino hydroxybenzoyl hexyl benzoate, diethylhexyl butamido triazone, ethylhexyl methoxycinnamate and mixtures thereof.

21. The method of claim 16, wherein the carrier is selected from the group consisting of water, alcohols, esters, butylene glycol, dipropylene glycol, ethanol, ethoxydiglycol, ethyl acetate, glycerol, propanol, isopropanol, macrogols, propyl propylene glycol(2) methyl ether, propyl propylene glycol(3) methyl ether, propylene carbonate, propylene glycol, triethylene glycol, isoparaffin, amyl acetate, amyl benzoate, benzyl acetate, butyl acetate, butylene glycol, butyl lactate, butooctyl benzoate, butooctylsalicylate, C10-C13 alkanes, C14-C17 alkanes, C11-C15 cycloalkanes, caprylyl butyrate, isoparaffins, diacetin, triacetin dicaprylyl ether, dicaprylyl maleate, and mixtures thereof.

22. The method of claim 16, wherein the components (a) and (b) are present in a weight ratio of approximately 1:99 to approximately 99:1.

23. The method of claim 22, wherein the components (a) and (b) are present in a weight ratio of approximately 5:95 to approximately 20:80.

24. The method of claim 22, wherein the components (a) and (b) are present in a weight ratio of approx. 10:90 to approx. 15:85.

25. The method of claim 16, wherein said composition comprises
(a) about 0.05 to about 19.5% b.w. of said at least one compound of Formula (I) 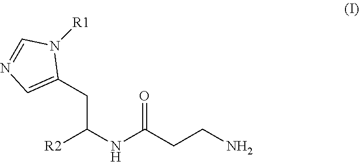
provided R1 denotes H or CH₃ and R2 denotes H or COOH or salts thereof,
(b) about 0.05 to about 19.5% b.w. of said at least one UV protection factor, and
(c) said carrier to 100% b.w.

26. The method of claim 25, wherein said composition consists essentially of said components (a), (b) and (c).

27. The method of claim 16, wherein said composition consists essentially of said components (a), (b) and (c).
